(19) United States
(12) Patent Application Publication
(10) Pub. No.: US 2011/0034457 A1
(43) Pub. Date: Feb. 10, 2011

(54) Pyridothiophene Compounds

(75) Inventors: Martin James Drysdale, Cambridge (GB); Brian William Dymock, Oxfordshire (GB); Xavier Barril-Alonso, Barcelona (ES)

Correspondence Address: BANNER & WITCOFF, LTD., 1100 13th STREET, N.W., SUITE 1200, WASHINGTON, DC, 20005-4051, US

(73) Assignee: VERNALIS (CAMBRIDGE) LIMITED., Abington (GB)

(21) Appl. No.: 12/909,014

(22) Filed: Oct. 21, 2010

Related U.S. Application Data
(63) Continuation of application No. 10/574,788, filed on Jan. 19, 2007, now abandoned.

(30) Foreign Application Priority Data
Oct. 10, 2003 (GB) 0323810.2

(51) Int. Cl. A61K 31/4365; C07D 471/04; A61K 31/496; A61K 31/5377; A61P 35/00; A61P 37/06; A61P 29/00

(52) U.S. Cl. 514/233.8; 546/114; 544/362; 544/127; 514/301; 514/253.04

(57) ABSTRACT

The use of compounds of formula (I) in therapy, particularly for the treatment of a disorder mediated by excessive or inappropriate HSP90 activity formula (I), wherein R₂ is a group of formula (IA):

—(Ar¹)ₘ-(Alk¹)ₚ-(Z)ᵣ-(Alk²)_{S}-Q (IA)

Ar¹, Alk¹, Z, Alk² and Q being as defined in the specification; m, p, r and s are independently 0 or 1; R₃ is hydrogen, an optional substituent, or an optionally substituted (C₁-C₆)alkyl, aryl or heteroaryl radical; and R₄ is a carboxylic ester, carboxamide or sulfonamide group; or a salt, N-oxide, hydrate, or solvate thereof.

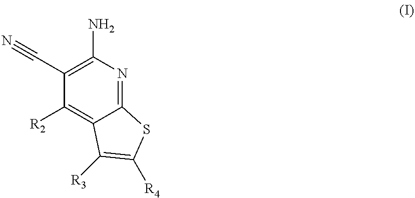

This invention relates to substituted bicyclic thieno[2,3-d]pyridine (herein referred to as ‘pyridothiophene’) compounds having HSP90 inhibitory activity, to the use of such compounds in medicine, in relation to diseases which are mediated by excessive or inappropriate HSP90 activity such as cancers, and to pharmaceutical compositions containing such compounds.

### BACKGROUND TO THE INVENTION

Molecular chaperones maintain the appropriate folding and conformation of proteins and are crucial in regulating the balance between protein synthesis and degradation. They have been shown to be important in regulating many important cellular functions, such as cell proliferation and apoptosis (Jolly and Morimoto, 2000; Smith et al., 1998; Smith, 2001).

### Heat Shock Proteins (HSPs)

Exposure of cells to a number of environmental stresses, including heat shock, alcohols, heavy metals and oxidative stress, results in the cellular accumulation of a number of chaperones, commonly known as heat shock proteins (HSPs). Induction of HSPs protects the cell against the initial stress insult, enhances recovery and leads to maintenance of a stress tolerant state. It has also become clear, however, that certain HSPs may also play a major molecular chaperone role under normal, stress-free conditions by regulating the correct folding, degradation, localization and function of a growing list of important cellular proteins.

A number of multigene families of HSPs exist, with individual gene products varying in cellular expression, function and localization. They are classified according to molecular weight, e.g., HSP70, HSP90, and HSP27. Several diseases in humans can be acquired as a result of protein misfolding (reviewed in Tytell et al., 2001; Smith et al., 1998). Hence the development of therapies which disrupt the molecular chaperone machinery may prove to be beneficial. In some conditions (e.g., Alzheimer's disease, prion diseases and Huntington's disease), misfolded proteins can cause protein aggregation resulting in neurodegenerative disorders. Also, misfolded proteins may result in loss of wild type protein function, leading to deregulated molecular and physiological functions in the cell.

HSPs have also been implicated in cancer. For example, there is evidence of differential expression of HSPs which may relate to the stage of tumour progression (Martin et al., 2000; Conroy et al., 1996; Kawanishi et al., 1999; Jameel et al., 1992; Hoang et al., 2000; Lebeau et al., 1991). As a result of the involvement of HSP90 in various critical oncogenic pathways and the discovery that certain natural products with anticancer activity are targeting this molecular chaperone, the fascinating new concept has been developed that inhibiting HSP function may be useful in the treatment of cancer. The first molecular chaperone inhibitor is currently undergoing clinical trials.

### HSP90

HSP90 constitutes about 1-2% of total cellular protein, and is usually present in the cell as a dimer in association with one of a number of other proteins (see, e.g., Pratt, 1997). It is essential for cell viability and it exhibits dual chaperone functions (Young et al., 2001). It plays a key role in the cellular stress response by interacting with many proteins after their native conformation has been altered by various environmental stresses, such as heat shock, ensuring adequate protein folding and preventing non-specific aggregation (Smith et al., 1998). In addition, recent results suggest that HSP90 may also play a role in buffering against the effects of mutation, presumably by correcting the inappropriate folding of mutant proteins (Rutherford and Lindquist, 1998). However, HSP90 also has an important regulatory role. Under normal physiological conditions, together with its endoplasmic reticulum homologue GRP94, HSP90 plays a housekeeping role in the cell, maintaining the conformational stability and maturation of several key client proteins. These can be subdivided into three groups: (a) steroid hormone receptors, (b) Ser/Thr or tyrosine kinases (e.g., ERBB2, RAF-1, CDK4, and LCK), and (c) a collection of apparently unrelated proteins, e.g., mutant p53 and the catalytic subunit of telomerase hTERT. All of these proteins play key regulatory roles in many physiological and biochemical processes in the cell. New HSP90 client proteins are continuously being identified.

The highly conserved HSP90 family in humans consists of four genes, namely the cytosolic HSP90a and HSP908 isoforms (Hickey et al., 1989), GRP94 in the endoplasmic reticulum (Argon et al., 1999) and FISP75/TRAP1 in the mitochondrial matrix (Felts et al., 2000). It is thought that all the family members have a similar mode of action, but bind to different client proteins depending on their localization within the cell. For example, ERBB2 is known to be a specific client protein of GRP94 (Argon et al., 1999) and type 1 tumour necrosis factor receptor (INFR1) and RB have both been shown to be clients of TRAP1 (Song et al., 1995; Chen et al., 1996).

HSP90 participates in a series of complex interactions with a range of client and regulatory proteins (Smith, 2001). Although the precise molecular details remain to be elucidated, biochemical and X-ray crystallographic studies (Prodromou et al., 1997; Stebbins et al., 1997) carried out over the last few years have provided increasingly detailed insights into the chaperone function of HSP90.

Following earlier controversy on this issue, it is now clear that HSP90 is an ATP-dependent molecular chaperone (Prodromou et al, 1997), with dimerization of the nucleotide binding domains being essential for ATP hydrolysis, which is in turn essential for chaperone function (Prodromou et al, 2000a). Binding of ATP results in the formation of a toroidal dimer structure in which the N terminal domains are brought into closer contact with each other resulting in a conformational switch known as the ‘clamp mechanism’ (Prodromou and Pearl, 2000b).

### Known HSP90 Inhibitors

The first class of HSP90 inhibitors to be discovered was the benzoquinone ansamycin class, which includes the compounds herbimycin A and geldanamycin. They were shown to reverse the malignant phenotype of fibroblasts transformed by the v-Src oncogene (Uehara et al., 1985), and subsequently to exhibit potent antitumour activity in both in vitro (Schulte et al., 1998) and in vivo animal models (Supko et al., 1995).

Immunoprecipitation and affinity matrix studies have shown that the major mechanism of action of geldanamycin involves binding to HSP90 (Whitesell et al., 1994; Schulte and Neckers, 1998). Moreover, X-ray crystallographic studies have shown that geldanamycin competes at the ATP binding site and inhibits the intrinsic ATPase activity of HSP90 (Prodromou et al., 1997; Panaretou et al., 1998). This in turn prevents the formation of mature multimeric HSP90 complexes capable of chaperoning client proteins. As a result, the client proteins are targeted for degradation via the ubiquitin proteasome pathway. 17-Allylamino, 17-demethoxygeldanamycin (17AAG) retains the property of HSP90 inhibition resulting in client protein depletion and antitumour activity in cell culture and xenograft models (Schulte et al, 1998; Kelland et al, 1999), but has significantly less hepatotoxicity than geldanamycin (Page et al, 1997). 17AAG is currently being evaluated in Phase I clinical trials.

Radicicol is a macrocyclic antibiotic shown to reverse the malignant phenotype of v-Src and v-Ha-Ras transformed fibroblasts (Kwon et al, 1992; Zhao et al, 1995). It was shown to degrade a number of signalling proteins as a consequence of HSP90 inhibition (Schulte et al., 1998). X-ray crystallographic data confirmed that radicicol also binds to the N terminal domain of HSP90 and inhibits the intrinsic ATPase activity (Roe et al., 1998). Radicicol lacks antitumour activity in vivo due to the unstable chemical nature of the compound.

Coumarin antibiotics are known to bind to bacterial DNA gyrase at an ATP binding site homologous to that of the HSP90. The coumarin, novobiocin, was shown to bind to the carboxy terminus of HSP90, i.e., at a different site to that occupied by the benzoquinone ansamycins and radicicol which bind at the N-terminus (Marcu et al., 2000b). However, this still resulted in inhibition of HSP90 function and degradation of a number of HSP90-chaperoned signalling proteins (Marcu et al., 2000a). Geldanamcyin cannot bind HSP90 subsequent to novobiocin; this suggests that some interaction between the N and C terminal domains must exist and is consistent with the view that both sites are important for HSP90 chaperone properties.

A purine-based HSP90 inhibitor, PU3, has been shown to result in the degradation of signalling molecules, including ERBB2, and to cause cell cycle arrest and differentiation in breast cancer cells (Chiosis et al., 2001).

### HSP90 as a Therapeutic Target

Due to its involvement in regulating a number of signalling pathways that are crucially important in driving the phenotype of a tumour, and the discovery that certain bioactive natural products exert their effects via HSP90 activity, the molecular chaperone HSP90 is currently being assessed as a new target for anticancer drug development (Neckers et al., 1999).

The predominant mechanism of action of geldanamycin, 17AAG, and radicicol involves binding to HSP90 at the ATP binding site located in the N-terminal domain of the protein, leading to inhibition of the intrinsic ATPase activity of HSP90 (see, e.g., Prodromou et al., 1997; Stebbins et al., 1997; Panaretou et al., 1998).

Inhibition of HSP90 ATPase activity prevents recruitment of co-chaperones and encourages the formation of a type of HSP90 heterocomplex from which these client proteins are targeted for degradation via the ubiquitin proteasome pathway (see, e.g., Neckers et al., 1999; Kelland et al., 1999).

Treatment with HSP90 inhibitors leads to selective degradation of important proteins involved in cell proliferation, cell cycle regulation and apoptosis, processes which are fundamentally important in cancer.

Inhibition of HSP90 function has been shown to cause selective degradation of important signalling proteins involved in cell proliferation, cell cycle regulation and apoptosis, processes which are fundamentally important and which are commonly deregulated in cancer (see, e.g., Hostein et al., 2001). An attractive rationale for developing drugs against this target for use in the clinic is that by simultaneously depleting proteins associated with the transformed phenotype, one may obtain a strong antitumour effect and achieve a therapeutic advantage against cancer versus normal cells. These events downstream of HSP90 inhibition are believed to be responsible for the antitumour activity of HSP90 inhibitors in cell culture and animal models (see, e.g., Schulte et al., 1998; Kelland et al., 1999).

### BRIEF DESCRIPTION OF THE INVENTION

The present invention relates to the use of a class of substituted thieno[2,3-d]pyridine compounds (referred to herein as pyridothiophenes) as HSP90 inhibitors, for example for inhibition of cancer cell proliferation. A core pyridothiophene ring system with amino, cyano and aromatic substitution on the pyrido ring are principle characterising features of the compounds with which the invention is concerned.

### DETAILED DESCRIPTION OF THE INVENTION

The present invention provides the use of a compound of formula (I), or a salt, N-oxide, hydrate, or solvate thereof, in the preparation of a composition for inhibition of HSP90 activity in vitro or in vivo:

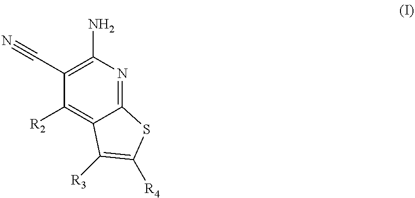

wherein
R₂ is a group of formula (IA):

—(Ar¹)ₘ-(Alk¹)ₚ-(Z)ᵣ-(Alk²)ₛ-Q (IA)
wherein in any compatible combination
   Ar¹ is an optionally substituted aryl or heteroaryl radical,
   Alk¹ and Alk² are optionally substituted divalent C₁-C₃ alkylene or C₂-C₃ alkenylene radicals,
   m, p, r and s are independently 0 or 1,
   Z is —O—, —S—, —(C═O)—, —(C═S)—, —SO₂—, —C(═O)O—, —C(═O)NR^{A}—, —C(═S)NR^{A}—, —SO₂NR^{A}—, —NR^{A}C(═O)—, —NR^{A}SO₂— or —NR^{A}— wherein R^{A} is hydrogen or C₁-C₆ alkyl, and
   Q is hydrogen or an optionally substituted carbocyclic or heterocyclic radical;
      R₃ is hydrogen, an optional substituent, or an optionally substituted (C₁-C₆)alkyl, aryl or heteroaryl radical; and
      R₄ is a carboxylic ester, carboxamide or sulfonamide group.

The invention also includes:

(i) A pharmaceutical or veterinary composition comprising a compound of formula (I) above, together with a pharmaceutically or veterinarily acceptable carrier.
(ii). A method of treatment of diseases or conditions mediated by excessive or inappropriate HSP90 activity in mammals which method comprises administering to the mammal an amount of a compound of formula (I) above effective to inhibit said HSP90 activity.

As used herein:
the term “carboxyl group” refers to a group of formula —COOH;
the term “carboxyl ester group” refers to a group of formula —COOR, wherein R is a radical actually or notionally derived from the hydroxyl compound ROH; and
the term “carboxamide group” refers to a group of formula —CONRₐR_{b}, wherein —NRₐR_{b} is a primary or secondary (including cyclic) amino group actually or notionally derived from ammonia or the amine HNRₐR_{b}.
the term “sulfonamide group” refers to a group of formula —SO₂NRₐR_{b}, wherein —NRₐR_{b} is a primary or secondary (including cyclic) amino group actually or notionally derived from ammonia or the amine HNRₐR_{b}

As used herein, the term “(C₁-C₆)alkyl” refers to a straight or branched chain alkyl radical having from 1 to 6 carbon atoms, including for example, methyl, ethyl, n-propyl, isopropyl, n-butyl, isobutyl, sec-butyl, t-butyl, n-pentyl and n-hexyl.

As used herein the term “divalent (C₁-C₆)alkylene radical” refers to a saturated hydrocarbon chain having from 1 to 6 carbon atoms and two unsatisfied valences.

As used herein, the term “(C₁-C₆)alkenyl” refers to a straight or branched chain alkenyl radical having from 2 to 6 carbon atoms and containing at least one double bond of E or Z configuration, including for example, ethenyl and allyl.

As used herein the term “divalent (C₂-C₆)alkenylene radical” refers to a hydrocarbon chain having from 2 to 6 carbon atoms, at least one double bond, and two unsatisfied valences.

As used herein the term “cycloalkyl” refers to a saturated carbocyclic radical having from 3-8 carbon atoms and includes, for example, cyclopropyl, cyclobutyl, cyclopentyl, cyclohexyl, cycloheptyl and cyclooctyl.

As used herein the term “cycloalkenyl” refers to a carbocyclic radical having from 3-8 carbon atoms containing at least one double bond, and includes, for example, cyclopentenyl, cyclohexenyl, cycloheptenyl and cyclooctenyl.

As used herein the term “aryl” refers to a mono-, bi- or tri-cyclic carbocyclic aromatic radical. Illustrative of such radicals are phenyl, biphenyl and napthyl.

As used herein the term “carbocyclic” refers to a cyclic radical whose ring atoms are all carbon, and includes monocyclic aryl, cycloalkyl, and cycloalkenyl radicals.

As used herein the term “heteroaryl” refers to a mono-, bi- or tri-cyclic aromatic radical containing one or more heteroatoms selected from S, N and O. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in particular refers to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with at least one substituent selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro and chloro), trifluoromethyl, trifluoromethoxy, nitro, nitrile (—CN), oxo, phenyl, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl group. The term “optional substituent” means one of the foregoing substituent groups.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

Some compounds with which the invention is concerned contain one or more actual or potential chiral centres because of the presence of asymmetric carbon atoms. The presence of several asymmetric carbon atoms gives rise to a number of diastereoisomers with R or S stereochemistry at each chiral centre. The invention includes the use of all such diastereoisomers and mixtures thereof.

### The Radical R₂

As stated, R₂ is a group of formula (IA):

—(Ar¹)ₘ-(Alk¹)ₚ-(Z)ᵣ-(Alk²)ₛ-Q (IA)

wherein in any compatible combination Ar¹ is an optionally substituted aryl or heteroaryl radical, Alk¹ and Alk² are optionally substituted divalent C₁-C₃ alkylene or C₂-C₃ alkenylene radicals, m, p, r and s are independently 0 or 1, Z is —O—, —S—, —(C═O)—, —(C═S)—, —SO₂—, —C(═O)O—, —C(═O)NR^{A}—, —C(═S)NR^{A}—, —SO₂NR^{A}—, —NR^{A}C(═O)—, —NR^{A}SO₂— or —NR^{A}— wherein R^{A} is hydrogen or C₁-C₆ alkyl, and Q is hydrogen or an optionally substituted carbocyclic or heterocyclic radical;

In a simple subclass of compounds with which the invention is concerned, m is 1 and each of p, r and s is 0, and Q is hydrogen, so that R₂ is optionally substituted aryl or heteroaryl. In such cases, R₂ may be, for example, optionally substituted phenyl, 2- or 3-thienyl, 2- or 3-furanyl, or 2-, 3- or 4-Pyridinyl. Currently preferred are compounds wherein R₂ is optionally substituted phenyl, for example where the optional substituents are selected from substituted methyl, ethyl, n- or isopropyl, methoxy, ethoxy, isopropoxy, chloro, or bromo, for example in the 4-position of the phenyl ring.

In other simple structures, m is 1, p, r and s are again each 0, and Q may be an optionally substituted carbocyclic or heterocyclic ring, for example phenyl, cyclohexyl, pyridyl, morpholino, piperidinyl, or piperazinyl ring. In such cases, Q is a direct substituent in the optionally substituted Ar¹ ring.

In more complex structures with which the invention is concerned, one or more of m, p, r and s may be 1, and Q may be hydrogen or an optionally substituted carbocyclic or heterocyclic ring. For example, p and/or s may be 1 and r may be 0, so that Q is linked to Ar¹ by an alkylene or alkenylene radical, for example a C₁-C₃ alkylene radical, which is optionally substituted. In other cases each of p, r, and s may be 1, in which cases, Q is linked to Ar¹ by an alkylene or alkenylene radical which is interrupted by the hetero atom-containing Z radical. In still other cases, p and s may be 0 and r may be 1, in which case Q is linked to Ar¹ via the hetero atom-containing Z radical.

Specific examples of R₁ groups of the above types are present in the compounds of the Examples herein.

### The Optional Substituent R₃

R₃ is hydrogen or an optional substituent, as defined above. Presently it is preferred that R₃ be amino(NH₂)

### The Group R₄

When R₄ is a carboxamide group, examples include those of formula —CONR^{B}(Alk)ₙR^{A} wherein
Alk is a divalent alkylene, alkenylene or alkynylene radical, for example a —CH₂—, —CH₂CH₂—, —CH₂CH₂CH₂—, —CH₂CH═CH—, or —CH₂CCCH₂-radical, and the Alk radical may be optionally substituted,
n is 0 or 1,
R^{B} is hydrogen or a C₁-C₆ alkyl or C₂-C₆ alkenyl group, for example methyl, ethyl, n- or iso-propyl, or allyl, (most preferably hydrogen)
R^{A} is hydroxy or optionally substituted carbocyclic, for example hydroxy and/or chloro-substituted phenyl and 3,4 methylenedioxyphenyl; or heterocyclyl, for example pyridyl, furyl, thienyl, N-piperazinyl, or N-morpholinyl any of which heterocyclic rings may be substituted,
or R^{A} and R^{B} taken together with the nitrogen to which they are attached form an N-heterocyclic ring which may optionally contain one or more additional hetero atoms selected from O, S and N, and which may optionally be substituted on one or more ring C or N atoms, examples of such N-heterocyclic rings including morpholino, piperidinyl, piperazinyl and N-phenylpiperazinyl.

When R₄ is a carboxylic ester group, examples include those of formula —COOR^{C} wherein R^{C} is a C₁-C₆ alkyl or C₂-C₆ alkenyl group, for example methyl, ethyl, n- or iso-propyl, or allyl; or an optionally substituted aryl or heteroaryl group, for example optionally substituted phenyl, pyridyl or thiazolyl; or an optionally substituted aryl(C₁-C₆ alkyl)- or heteroaryl(C₁-C₆ alkyl)-group such as benzyl or pyridylmethyl; or an optionally substituted cycloalkyl group such as cyclopentyl or cyclohexyl.

Specific compounds with which the invention is concerned include those of the Examples.

Compounds with which the invention is concerned may be prepared by literature methods, such as those of the preparative Examples herein, and methods analogous thereto. For example the following general reaction scheme can be employed:

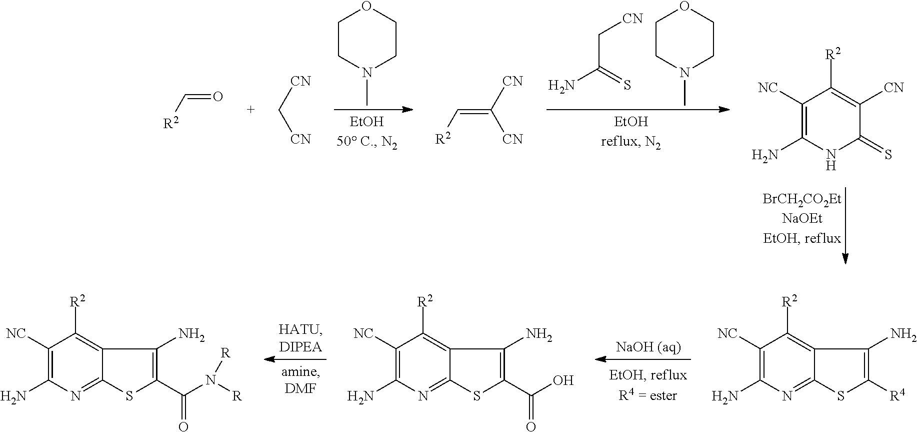

Starting material are either available commercially or can be made according to literature methods. Subsequent reactions may be carried out on R², R³ or R⁴ to prepare additional compounds of formula (I)

The compounds with which the invention is concerned are inhibitors of HSP90 and are useful in the treatment of diseases which are mediated by excessive or inappropriate HSP90 activity such as cancers; viral diseases such as Hepatitis C(HCV) (Waxman, 2002); Immunosupression such as in transplantation (Bijlmakers, 2000 and Yorgin, 2000); Anti-inflammatory diseases (Bucci, 2000) such as Rheumatoid arthritis, Asthma, MS, Type I Diabetes, Lupus, Psoriasis and Inflammatory Bowel Disease; Cystic fibrosis (Fuller, 2000); Angiogenesis-related diseases (Hur, 2002 and Kurebayashi, 2001): diabetic retinopathy, haemangiomas, psoriasis, endometriosis and tumour angiogenesis. Also an Hsp90 inhibitor of the invention may protect normal cells against chemotherapy-induced toxicity and be useful in diseases where failure to undergo apoptosis is an underlying factor. Such an Hsp90 inhibitor may also be useful in diseases where the induction of a cell stress or heat shock protein response could be beneficial, for example, protection from hypoxia-ischemic injury due to elevation of Hsp70 in the heart (Hutter, 1996 and Trost, 1998) and brain (Plumier, 1997 and Rajder, 2000). An Hsp90 inhibitor-induced increase in Hsp70 levels could also be useful in diseases where protein misfolding or aggregation is a major causal factor, for example, neurogenerative disorders such as scrapie/CJD, Huntingdon's and Alzheimer's (Sittler, 2001; Trazelt, 1995 and Winklhofer, 2001)”.

It will be understood that the specific dose level for any particular patient will depend upon a variety of factors including the activity of the specific compound employed, the age, body weight, general health, sex, diet, time of administration, route of administration, rate of excretion, drug combination and the causative mechanism and severity of the particular disease undergoing therapy. In general, a suitable dose for orally administrable formulations will usually be in the range of 0.1 to 3000 mg once, twice or three times per day, or the equivalent daily amount administered by infusion or other routes. However, optimum dose levels and frequency of dosing will be determined by clinical trials as is conventional in the art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The following examples illustrate the preparation and activities of specific compounds with which the invention is concerned.

### Example 1

### 3,6-Diamino-5-cyano-4-phenyl-thieno[2,3-b]pyridine-2-carboxylic acid ethylamide

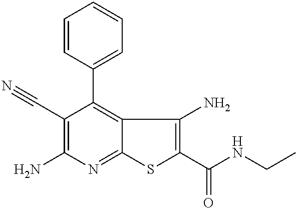

### Step 1

Malonitrile (1 eq) was dissolved in EtOH with stirring under a N₂ atmosphere. Benzaldehyde (1.1 eq) was added followed by catalytic N-methylmorpholine. The mixture was stirred at 50° C. for 30 minutes, after which time the solution had produced a heavy precipitate. The reaction was allowed to cool to room temperature and 2-benzylidene-malononitrile was collected by vacuum filtration.

LC retention time=2.416 min [M+H]⁺ not observed

### Step 2

2-Benzylidene-malononitrile (1 eq) was dissolved with stirring in EtOH and cyanothioacetamide (1 eq) was added. N-Methylmorpholine (2 eq) was added and all was heated at reflux overnight under a N₂ atmosphere. Allowed to cool to room temperature, and the resulting precipitate, 6-amino-4-phenyl-2-thioxo-1,2-dihydro-pyridine-3,5-dicarbonitrile, was collected by vacuum filtration. LC retention time=2.04 min [M+H]⁺ 253.1

### Step 3

6-Amino-4-phenyl-2-thioxo-1,2-dihydro-pyridine-3,5-dicarbonitrile (leg) was added to a freshly prepared solution of NaOEt (3 eq) in EtOH. Ethylbromoacetate (1 eq) was added and stirred for a few minutes at room temperature during which time, a thick solid precipitated out of solution. The reaction mixture was heated at 80° C. for 1.5 hours, after which time the solution was cooled back to room temperature. The resulting precipitate, 3,6-diamino-5-cyano-4-phenyl-thieno[2,3-b]pyridine-2-carboxylic acid ethyl ester, was collected by vacuum filtration.

LC retention time=2.603 min [M+H]⁺ 339.1

### Step 4

3,6-Diamino-5-cyano-4-phenyl-thieno[2,3-b]pyridine-2-carboxylic acid ethyl ester was suspended in EtOH and 3M NaOH (aq) (3 eq) was added. The suspension was heated to reflux and dissolution occurred. Allowed to cool to room temperature on completion and the reaction mixture was concentrated in vacuo. The residue was taken up in water and acidified by addition of 1M HCl (aq). The resulting precipitate, 3,6-diamino-5-cyano-4-phenyl-thieno[2,3-b]pyridine-2-carboxylic acid, was collected by vacuum filtration. LC retention time=2.098 min [M+H]⁺ 311.1

### Step 5

3,6-Diamino-5-cyano-4-phenyl-thieno[2,3-b]pyridine-2-carboxylic acid (leg) was taken up in DMF with stirring and HATU (1.1 eq), excess 2M methanolic ethylamine and DIPEA (3 eq) were added. Stirred overnight at room temperature. The DMF was removed by evaporation and the residue was dissolved in DCM. This was washed with 1M HCl (aq), saturated NaHCO₃ (aq) and brine. The organic phase was dried over MgSO₄, filtered, and concentrated in vacuo. The crude product was then purified by column chromatography, eluting with 0.5% MeOH/DCM to give 3,6-diamino-5-cyano-4-phenyl-thieno[2,3-b]pyridine-2-carboxylic acid ethylamide.

LC retention time=2.259 min [M+H]⁺ 338.1

The compound of Example 1 had activity in the “A” range when tested in the fluorescence polarisation assay described below.

Additional compounds were either procured from commercially available sources, or synthesised by methods analogous to that of Example 1. Compounds procured from commercial sources may also be synthesised by the method of Example 1. Additional compounds are identified and characterised by mass ion data in the following Table. The source of the compound is identified in the final column, or the compound by the following

### Key:

“Synth” compound synthesised de novo.
“Specs” compound purchased from: Specs, Fleminglaan 16, 2289 CP Rijswijk, The Netherlands
“IBS” compound purchased from: InterBioScreen Ltd., 121019 Moscow, P.O. Box 218, Russia
“ASI” compound purchased from: ASINEX Ltd, 6 Schukinskaya Street, Moscow 123182, Russia

The penultimate column of the table shows the result obtained in the fluorescence polarisation assay described below

| | | | Hsp90 FP | |
|---|---|---|---|---|
| Example | Structure | MH+ | IC50 | Source |
| 2 | 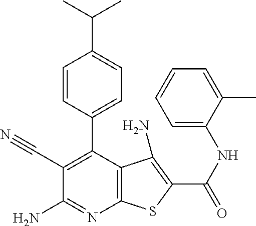 | 442 | B | Specs |
| 3 | 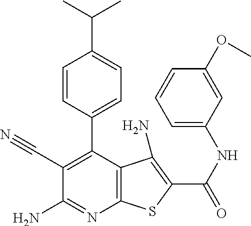 | 458 | B | Specs |
| 4 | 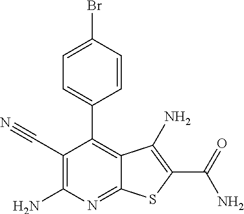 | 389 | B | IBS |
| 5 | 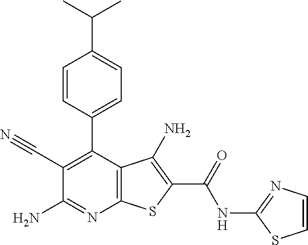 | 435 | B | IBS |
| 6 | 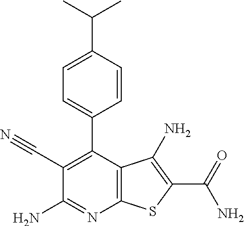 | 352 | A | IBS |
| 7 | 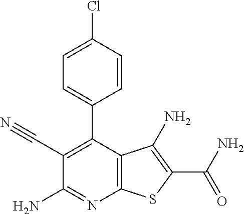 | 344 | A | IBS |
| 8 | 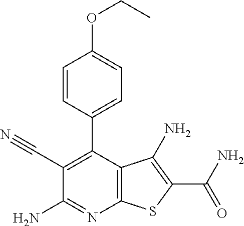 | 354 | A | ASI |
| 9 | 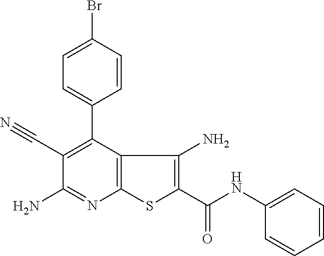 | 465 | B | IBS |
| 10 | 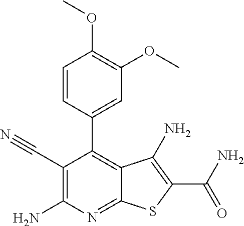 | 370 | B | IBS |
| 11 | 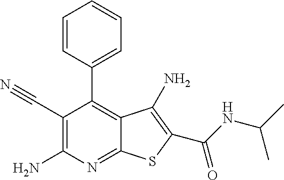 | 352 | B | Synth |
| 12 | 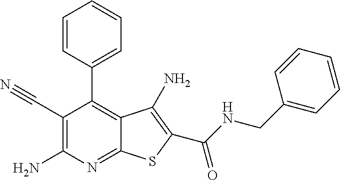 | 400 | B | Synth |
| 13 | 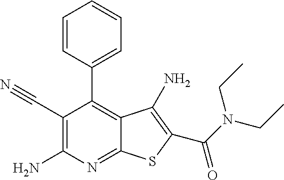 | 366 | B | Synth |
| 14 | 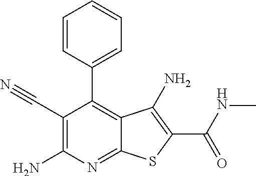 | 324 | A | Synth |
| 15 | 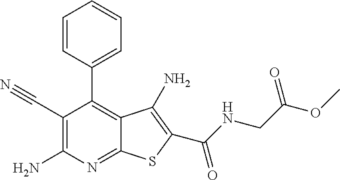 | 382 | A | Synth |
| 16 | 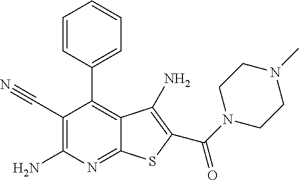 | 393 | B | Synth |
| 17 | 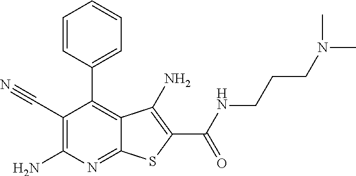 | 395 | B | Synth |
| 18 | 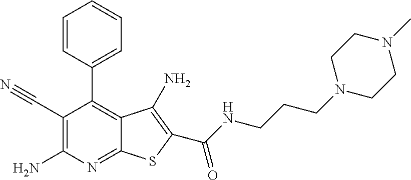 | 450 | B | Synth |
| 19 | 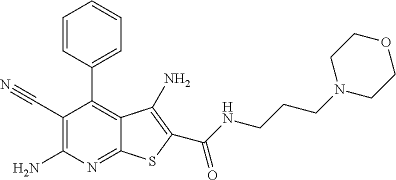 | 437 | B | Synth |
| 20 | 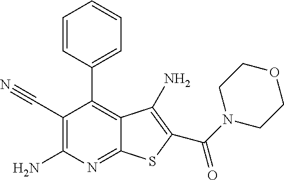 | 380 | B | Synth |
| 21 | 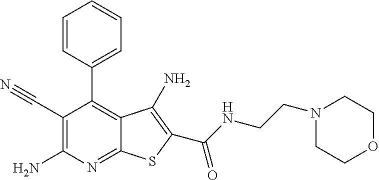 | 422 | B | Synth |
| 22 | 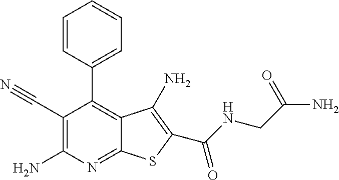 | 367 | A | Synth |
| 23 | 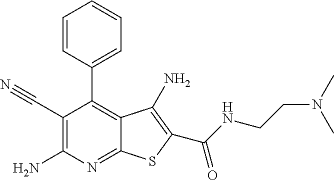 | 381 | B | Synth |
| 24 | 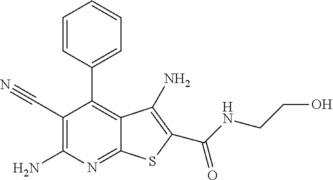 | 354 | B | Synth |
| 25 | 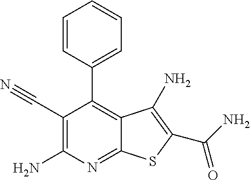 | 310 | B | Synth |
| 26 | 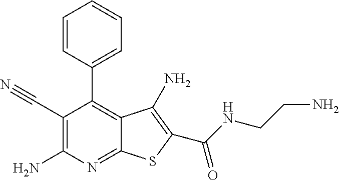 | 352 | B | Synth |
| 27 | 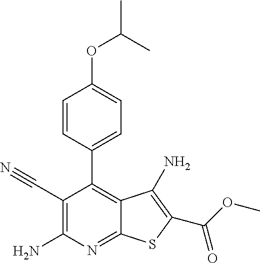 | 383 | B | Synth |

### Fluorescence Polarization Assay

Fluorescence polarization {also known as fluorescence anisotropy} measures the rotation of a fluorescing species in solution, where the larger molecule the more polarized the fluorescence emission. When the fluorophore is excited with polarized light, the emitted light is also polarized. The molecular size is proportional to the polarization of the fluorescence emission.

The fluoroscein-labelled probe—RBT0045864-FAM—

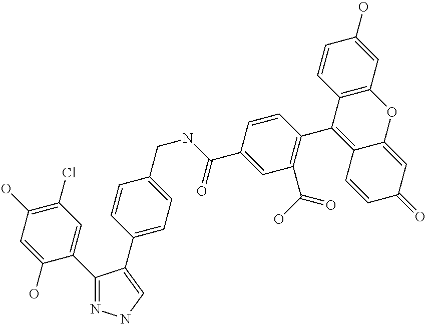

binds to HSP90 {full-length human, full-length yeast or N-terminal domain HSP90} and the anisotropy {rotation of the probe:protein complex} is measured.

Test compound is added to the assay plate, left to equilibrate and the anisotropy measured again. Any change in anisotropy is due to competitive binding of compound to HSP90, thereby releasing probe.

### Materials

Chemicals are of the highest purity commercially available and all aqueous solutions are made up in AR water.
1) Costar 96-well black assay plate #3915
2) Assay buffer of (a) 100 mM Tris pH7.4; (b) 20 mM KCl; (c) 6 mM MgCl₂. Stored at room temperature.
3) BSA (bovine serum albumen) 10 mg/ml (New England Biolabs # B9001S)
4) 20 mM probe in 100% DMSO stock concentration. Stored in the dark at RT. Working concentration is 200 nM diluted in AR water and stored at 4° C. Final concentration in assay 80 nM.
5) E. coli expressed human full-length HSP90 protein, purified >95% (see, e.g., Panaretou et al., 1998) and stored in 50 μL aliquots at −80° C.

### Protocol

1) Add 100 μl 1× buffer to wells 11A and 12A (=FP BLNK)
2) Prepare assay mix—all reagents are kept on ice with a lid on the bucket as the probe is light-sensitive.

| | |
|---|---|
| I. | Final Concⁿ |

| 1x Hsp90 FP Buffer | 10 | ml | 1x | |
|---|---|---|---|---|
| BSA 10 mg/ml (NEB) | 5.0 | μl | 5 | μg/ml |
| Probe 200 μM | 4.0 | μl | 80 | nM |
| Human full-length Hsp90 | 6.25 | μl | 200 | nM |

3) Aliquot 100 μl assay mix to all other wells
4) Seal plate and leave in dark at room temp for 20 minutes to equilibrate

### Compound Dilution Plate—1×3 Dilution Series

1) In a clear 96-well v-bottom plate—{# VWR 007/0081257} add 10 μl 100% DMSO to wells B1 to H11
2) To wells A1 to A11 add 17.5 μl 100% DMSO
3) Add 2.5 μl cpd to A1. This gives 2.5 mM {50×} stock cpd—assuming cpds 20 mM.
4) Repeat for wells A2 to A10. Control in columns 11 and 12.
5) Transfer 5 μl from row A to row B—not column 12. Mix well.
6) Transfer 5 μl from row B to row C. Mix well.
7) Repeat to row G.
8) Do not add any compound to row H—this is the 0 row.
9) This produces a 1×3 dilution series from 50 μM to 0.07 μM.
10) In well B12 prepare 20 μl of 100 μM standard compound.
11) After first incubation the assay plate is read on a Fusion™ a-FP plate reader (Packard BioScience, Pangbourne, Berkshire, UK).
12) After the first read, 2 μl of diluted compound is added to each well for columns 1 to 10. In column 11 {provides standard curve} only add compound B11-H11. Add 2 μl of 100 mM standard cpd to wells B12-H12 {is positive control}
13) The Z′ factor is calculated from zero controls and positive wells. It typically gives a value of 0.7-0.9.

The compounds tested in the above assay were assigned to one of two activity ranges, namely A=<10 μM; B=>10 μM, and those assignments are reported above.

### REFERENCES

A number of publications are cited above in order to more fully describe and disclose the invention and the state of the art to which the invention pertains. Full citations for these references are provided below. Each of these references is incorporated herein by reference in its entirety into the present disclosure.
Argon Y and Simen B B. 1999 “Grp94, an ER chaperone with protein and peptide binding properties”, Semin. Cell Dev. Biol., Vol. 10, pp. 495-505.
Bijlmakers M-JJE, Marsh M. 2000 “Hsp90 is essential for the synthesis and subsequent membrane association, but not the maintenance, of the Src-kinase p56lck”, Molecular Biology of the Cell, Vol. 11(5), pp. 1585-1595.
Bucci M; Roviezzo F; Cicala C; Sessa W C, Cirino G. 2000 “Geldanamycin, an inhibitor of heat shock protein 90 (Hsp90) mediated signal transduction has anti-inflammatory effects and interacts with glucocorticoid receptor in vivo”, Brit. J. Pharmacol., Vol 131(1), pp. 13-16.
Chen C-F, Chen Y, Dai K D, Chen P-L, Riley D J and Lee W-H. 1996 “A new member of the hsp90 family of molecular chaperones interacts with the retinoblastoma protein during mitosis and after heat shock”, Mol. Cell. Biol., Vol. 16, pp. 4691-4699.
Chiosis G, Timaul M N, Lucas B, Munster P N, Zheng F F, Sepp-Lozenzino L and Rosen N. 2001 “A small molecule designed to bind to the adenine nucleotide pocket of HSP90 causes Her2 degradation and the growth arrest and differentiation of breast cancer cells”, Chem. Biol., Vol. 8, pp. 289-299.
Conroy S E and Latchman D S. 1996 “Do heat shock proteins have a role in breast cancer?”, Brit. J. Cancer, Vol. 74, pp. 717-721.
Felts S J, Owen B A L, Nguyen P, Trepel J, Donner D B and Toft D O. 2000 “The HSP90-related protein TRAP1 is a mitochondrial protein with distinct functional properties”, J. Biol. Chem., Vol. 5, pp. 3305-3312.
Fuller W, Cuthbert A W. 2000 “Post-translational disruption of the delta F508 cystic fibrosis transmembrane conductance regulator (CFTR)-molecular Chaperone complex with geldanamycin stabilizes delta F508 CFTR in the rabbit reticulocyte lysate”, J. Biol. Chem.; Vol 275(48), pp. 37462-37468.
Hickey E, Brandon S E, Smale G, Lloyd D and Weber L A. 1999 “Sequence and regulation of a gene encoding a human 89-kilodalton heat shock protein”, Mol. Cell. Biol., Vol. 9, pp. 2615-2626.
Hoang A T, Huang J, Rudra-Gonguly N, Zheng J, Powell W C, Rabindron S K, Wu C and Roy-Burman P. 2000 “A novel association between the human heat shock transcription factor I (HSF1) and prostate adenocarcinoma, Am. J. Pathol., Vol. 156, pp. 857-864.
Hostein I, Robertson D, Di Stefano F, Workman P and Clarke P A. 2001 “Inhibition of signal transduction by the HSP90 inhibitor 17-allylamino-17-demethoxygeldanamycin results in cytostasis and apoptosis”, Cancer Res., Vol. 61, pp. 4003-4009.
Hur E, Kim H-H, Choi S M, Kim J H, Yim S, Kwon H J, Choi Y, Kim D K, Lee M-O, Park H. 2002 “Reduction of hypoxia-induced transcription through the repression of hypoxia-inducible factor-1α/aryl hydrocarbon receptor nuclear translocator DNA binding by the 90-kDa heat-shock protein inhibitor radicicol”, Mol. Pharmacol., Vol 62(5), pp. 975-982.
Hutter et al, 1996, Circulation, Vol. 94, pp. 1408.
Jameel A, Skilton R A, Campbell T A, Chander S K, Coombes R C and Luqmani Y A. 1992 “Clinical and biological significance of HSP89a in human breast cancer”, Int. J. Cancer, Vol. 50, pp. 409-415.
Jolly C and Morimoto R I. 2000 “Role of the heat shock response and molecular chaperones in oncogenesis and cell death”, J. Natl. Cancer Inst., Vol. 92, pp. 1564-1572.
Kawanishi K, Shiozaki H, Doki Y, Sakita I, Inoue M, Yano M, Tsujinata T, Shamma A and Monden M. 1999 “Prognostic significance of heat shock proteins 27 and 70 in patients with squamous cell carcinoma of the esophagus”, Cancer, Vol. 85, pp. 1649-1657.
Kelland L R, Abel G, McKeage M J, Jones M, Goddard P M, Valenti M, Murrer B A and Harrap K R. 1993 “Preclinical antitumour evaluation of bis-acetalo-amino-dichloro-cyclohexylamine platinum (IV): an orally active platinum drug”, Cancer Research, Vol. 53, pp. 2581-2586.
Kelland L R, Sharp S Y, Rogers P M, Myers T G and Workman P. 1999 “DT-diaphorase expression and tumor cell sensitivity to 17-allylamino, 17-demethoxygeldanamycin, an inhibitor of heat shock protein 90”, J. Natl. Cancer Inst., Vol. 91, pp. 1940-1949.
Kurebayashi J, Otsuki T, Kurosumi M, Soga S, Akinaga S, Sonoo, H. 2001 “A radicicol derivative, KF58333, inhibits expression of hypoxia-inducible factor-1α and vascular endothelial growth factor, angiogenesis and growth of human breast cancer xenografts”, Jap. J. Cancer Res., Vol 92(12), 1342-1351.
Kwon H J, Yoshida M, Abe K, Horinouchi S and Bepple T. 1992 “Radicicol, an agent inducing the reversal of transformed phentoype of src-transformed fibroblasts, Biosci., Biotechnol., Biochem., Vol. 56, pp. 538-539.
Lebeau J, Le Cholony C, Prosperi M T and Goubin G. 1991 “Constitutive overexpression of 89 kDa heat shock protein gene in the HBL100 mammary cell line converted to a tumorigenic phenotype by the EJ/T24 Harvey-ras oncogene”, Oncogene, Vol. 6, pp. 1125-1132.
Marcu M G, Chadli A, Bouhouche I, Catelli M and Neckers L. 2000a “The heat shock protein 90 antagonist novobiocin interacts with a previously unrecognized ATP-binding domain in the carboxyl terminus of the chaperone”, J. Biol. Chem., Vol. 275, pp. 37181-37186.
Marcu M G, Schulte T W and Neckers L. 2000b “Novobiocin and related coumarins and depletion of heat shock protein 90-dependent signaling proteins”, J. Natl. Cancer Inst., Vol. 92, pp. 242-248.
Martin K J, Kritzman B M, Price L M, Koh B, Kwan C P, Zhang X, MacKay A, O'Hare M J, Kaelin C M, Mutter G L, Pardee A B and Sager R. 2000 “Linking gene expression patterns to therapeutic groups in breast cancer”, Cancer Res., Vol. 60, pp. 2232-2238.
Neckers L, Schulte T W and Momnaaugh E. 1999 “Geldanamycin as a potential anti-cancer agent: its molecular target and biochemical activity”, Invest. New Drugs, Vol. 17, pp. 361-373,
Page J, Heath J, Fulton R, Yalkowsky E, Tabibi E, Tomaszewski J, Smith A and Rodman L. 1997 “Comparison of geldanamycin (NSC-122750) and 17-allylaminogeldanamycin (NSC-330507D) toxicity in rats”, Proc. Am. Assoc. Cancer Res., Vol. 38, pp. 308.
Panaretou B, Prodromou C, Roe S M, O'Brien R, Ladbury J E, Piper P W and Pearl L H. 1998 “ATP binding and hydrolysis are essential to the function of the HSP90 molecular chaperone in vivo”, EMBO J., Vol. 17, pp. 4829-4836.
Plumier et al, 1997, Cell. Stress Chap., Vol. 2, pp. 162
Pratt W B. 1997 “The role of the HSP90-based chaperone system in signal transduction by nuclear receptors and receptors signalling via MAP kinase”, Annu. Rev. Pharmacol. Toxicol., Vol. 37, pp. 297-326.
Prodromou C and Pearl L H. 2000a “Structure and in vivo function of HSP90”, Curr. Opin. Struct. Biol., Vol. 10, pp. 46-51.
Prodromou C, Roe S M, O'Brien R, Ladbury J E, Piper P W and Pearl L H. 1997 “Identification and structural characterization of the ATP/ADP-binding site in the HSP90 molecular chaperone”, Cell, Vol. 90, pp. 65-75.
Prodromou C, Panaretou B, Chohan S, Siligardi G, O'Brien R, Ladbury J E, Roe S M, Piper P W and Pearl L H. 2000b “The ATPase cycle of HSP90 drives a molecular ‘clamp’ via transient dimerization of the N-terminal domains”, EMBO J., Vol. 19, pp. 4383-4392.
Rajder et al, 2000, Ann. Neurol., Vol. 47, pp. 782.
Roe S M, Prodromou C, O'Brien R, Ladbury J E, Piper P W and Pearl L H. 1999 “Structural basis for inhibition of the HSP90 molecular chaperone by the antitumour antibiotics radicicol and geldanamycin”, J. Med. Chem., Vol. 42, pp. 260-266.
Rutherford S L and Lindquist S. 1998 “HSP90 as a capacitor for morphological evolution. Nature, Vol. 396, pp. 336-342.
Schulte T W, Akinaga S, Murakata T, Agatsuma T, Sugimoto S, Nakano H, Lee Y S, Simen B B, Argon Y, Felts S, Toft D O, Neckers L M and Sharma S V. 1999 “Interaction of radicicol with members of the heat shock protein 90 family of molecular chaperones”, Mol. Endocrinology, Vol. 13, pp. 1435-1448.
Schulte T W, Akinaga S, Soga S, Sullivan W, Sensgard B, Toft D and Neckers L M. 1998 “Antibiotic radicicol binds to the N-terminal domain of HSP90 and shares important biologic activities with geldanamcyin”, Cell Stress and Chaperones, Vol. 3, pp. 100-108.
Schulte T W and Neckers L M. 1998 “The benzoquinone ansamycin 17-allylamino-17-deemthoxygeldanamcyin binds to HSP90 and shares important biologic activities with geldanamycin”, Cancer Chemother. Pharmacol., Vol. 42, pp. 273-279.
Sittler et al, 2001, Hum. Mol. Genet., Vol. 10, pp. 1307.
Smith D F. 2001 “Chaperones in signal transduction”, in: Molecular chaperones in the cell (P Lund, ed.; Oxford University Press, Oxford and NY), pp. 165-178.
Smith D F, Whitesell L and Katsanis E. 1998 “Molecular chaperones: Biology and prospects for pharmacological intervention”, Pharmacological Reviews, Vol. 50, pp. 493-513.
Song H Y, Dunbar J D, Zhang Y X, Guo D and Donner D B. 1995 “Identification of a protein with homology to hsp90 that binds the type 1 tumour necrosis factor receptor”, J. Biol. Chem., Vol. 270, pp. 3574-3581.
Stebbins C E, Russo A, Schneider C, Rosen N, Hartl F U and Pavletich N P. 1997 “Crystal structure of an HSP90-geldanamcyin complex: targeting of a protein chaperone by an antitumor agent”, Cell, Vol. 89, pp. 239-250.
Supko J G, Hickman R L, Greyer M R and Malspeis L. 1995 “Preclinical pharmacologic evaluation of geldanamycin as an antitumour agent”, Cancer Chemother. Pharmacol., Vol. 36, pp. 305-315.
Tratzelt et al, 1995, Proc. Nat. Acad. Sci., Vol. 92, pp. 2944.
Trost et al, 1998, J. Clin. Invest., Vol. 101, pp. 855.
Tytell M and Hooper P L. 2001 “Heat shock proteins: new keys to the development of cytoprotective therapies”, Emerging Therapeutic Targets, Vol. 5, pp. 267-287.
Uehara U, Hod M, Takeuchi T and Umezawa H.1986 “Phenotypic change from transformed to normal induced by benzoquinoid ansamycins accompanies inactivation of p60src in rat kidney cells infected with Rous sarcoma virus”, Mol. Cell. Biol., Vol. 6, pp. 2198-2206.
Waxman, Lloyd H. Inhibiting hepatitis C virus processing and replication. (Merck & Co., Inc., USA). PCT Int. Appl. (2002), WO 0207761
Winklhofer et al, 2001, J. Biol. Chem., Vol. 276, 45160.
Whitesell L, Mimnaugh E G, De Costa B, Myers C E and Neckers L M. 1994 “Inhibition of heat shock protein HSP90-pp 60v-src heteroprotein complex formation by benzoquinone ansamycins: essential role for stress proteins in oncogenic transformation”, Proc. Natl. Acad. Sci. USA., Vol. 91, pp. 8324-8328.
Yorgin et al. 2000 “Effects of geldanamycin, a heat-shock protein 90-binding agent, on T cell function and T cell nonreceptor protein tyrosine kinases”, J. Immunol., Vol 164(6), pp. 2915-2923.
Young J C, Moarefi I and Hartl F U. 2001 “HSP90: a specialized but essential protein-folding tool”, J. Cell. Biol., Vol. 154, pp. 267-273.
Zhao J F, Nakano H and Sharma S. 1995 “Suppression of RAS and MOS transformation by radicicol”, Oncogene, Vol. 11, pp. 161-173.

1-11. (canceled)

12. A method of inhibiting HSP90 activity in mammals which method comprises administering to the mammal an amount of a compound of formula (I), or a salt or N-oxide thereof, effective to inhibit said HSP90 activity: 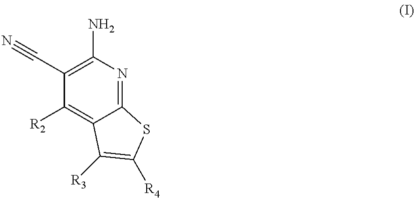
wherein
R² is a group of formula (IA):
—(Ar¹)ₘ-(Alk¹)ₚ-(Z)ᵣ-(Alk²)ₛ-Q (IA)
wherein in any compatible combination
Ar¹ is an optionally substituted aryl or heteroaryl radical,
Alk¹ and Alk² are optionally substituted divalent C₁-C₃ alkylene or C₂-C₃ alkenylene radicals,
m, p, r and s are independently 0 or 1,
Z is —O—, —S—, —(C═O)—, —(C═S)—SO₂—, —C(═O)O—, —C(═O)NR^{A}—, —C(═S)NR^{A}—, —SO₂NR^{A}—, —NR^{A}C(═O)—, —NR^{A}SO₂— or —NR^{A}—
wherein R^{A} is hydrogen or C₁-C₆ alkyl, and
Q is hydrogen or an optionally substituted carbocyclic or heterocyclic radical;
R₃ is hydrogen, an optional substituent, or an optionally substituted (C₁C₆)alkyl, aryl or heteroaryl radical; and
R₄ is a carboxamide or sulfonamide group,
wherein the optional substituent is selected from the group consisting of: C₁-C₆ alkyl, C₁-C₆ alkoxy, hydroxyl, hydroxy C₁-C₆ alkyl, mercapto, mercapto C₁-C₆ alkyl, C₁-C₆ alkylthio, halo, trifluoromethyl, trifluoromethoxy, nitro, nitrile (—CN), oxo, phenyl, —COOH, COOR^{C}, —COR^{C}, —SO₂R^{C}, —CONH₂, —SO₂NH₂, —CONHR^{C}, —SO₂NHR^{C}, —CONR^{C}R^{D}, —SO₂NR^{C}R^{D}, —NH₂, —NHR^{C}, —NR^{C}R^{D}, —OCONH₂, —OCONHR^{C}, —OCONR^{C}R^{D}, —NHCOR^{C}, —NHCOOR^{C}, —NHR^{D}COOR^{C}, —NHSO₂OR^{C}, —NR^{D}SO₂OR^{C}, —NHCONH₂, —NR^{C}CONH₂, —NHCONHR^{D}, —NR^{C}CONHR^{D}, —NHCONHR^{C}R^{D}, and —NR^{C}CONR^{C}R^{D}, wherein R^{C} and R^{D} are independently C₁-C₆ alkyl groups.

13-14. (canceled)

15. A pharmaceutical or veterinary composition comprising a compound of formula (I), or a salt or N-oxide thereof: 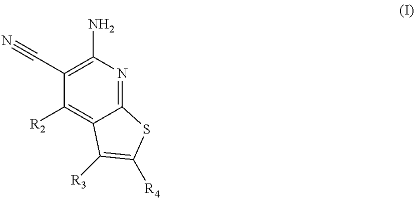
wherein
R² is a group of formula (IA):
—(Ar¹)ₘ-(Alk¹)ₚ-(Z)ᵣ-(Alk²)ₛ-Q (IA)
wherein in any compatible combination
Ar¹ is an optionally substituted aryl or heteroaryl radical,
Alk¹ and Alk² are optionally substituted divalent C₁-C₃ alkylene or C₂-C₃ alkenylene radicals,
m, p, r and s are independently 0 or 1,
Z is —O—, —S—, —(C═O)—, —(C═S)—SO₂—, —C(═O)O—, —C(═O)NR^{A}—, —C(═S)NR^{A}—, —SO₂NR^{A}—, —NR^{A}C(═O)—, —NR^{A}SO₂— or —NR^{A}—
wherein R^{A} is hydrogen or C₁-C₆ alkyl, and
Q is hydrogen or an optionally substituted carbocyclic or heterocyclic radical;
R₃ is hydrogen, an optional substituent, or an optionally substituted (C₁C₆)alkyl, aryl or heteroaryl radical; and
R₄ is a carboxamide or sulfonamide group,
wherein the optional substituent is selected from the group consisting of: C₁-C₆ alkyl, C₁-C₆ alkoxy, hydroxyl, hydroxy C₁-C₆ alkyl, mercapto, mercapto C₁-C₆ alkyl, C₁-C₆ alkylthio, halo, trifluoromethyl, trifluoromethoxy, nitro, nitrile (—CN), oxo, phenyl, —COOH, COOR^{C}, —COR^{C}, —SO₂R^{C}, —CONH₂—SO₂NH₂, —CONHR^{C}, —SO₂NHR^{C}, —CONR^{C}R^{D}, —SO₂NR^{C}R^{D}, —NH₂, —NHR^{C}, —NR^{C}R^{D}, —OCONH₂, —OCONHR^{C}, —OCONR^{C}R^{D}, —NHCOR^{C}, —NHCOOR^{C}, —NHR^{D}COOR^{C}, —NHSO₂OR^{C}, —NR^{D}SO₂OR^{C}, —NHCONH₂, —NR^{C}CONH₂, —NHCONHR^{D}, —NR^{C}CONHR^{D}, —NHCONHR^{C}R^{D}, and —NR^{C}CONR^{C}R^{D}, wherein R^{C} and R^{D} are independently C₁-C₆ alkyl groups,
in an amount effective to inhibit said HSP90 activity together with a pharmaceutically or veterinarily acceptable carrier.

16.-20. (canceled)

21. The method of claim 12 wherein m is 1, each of p, r and s is 0, and Q is hydrogen.

22. The method of claim 21 wherein R₂ is optionally substituted phenyl, 2- or 3-thienyl, 2- or 3-furanyl, or 2-, 3- or 4-pyridinyl,
wherein the optional substituent is selected from the group consisting of: C₁-C₆ alkyl, C₁-C₆ alkoxy, hydroxyl, hydroxy C₁-C₆ alkyl, mercapto, mercapto C₁-C₆ alkyl, C₁-C₆ alkylthio, halo, trifluoromethyl, trifluoromethoxy, nitro, nitrile (—CN), oxo, phenyl, —COOH, COOR^{C}, —COR^{C}, —SO₂R^{C}, —CONH₂, —SO₂NH₂, —CONHR^{C}, —SO₂NHR^{C}, —CONR^{C}R^{D}, —SO₂NR^{C}R^{D}, —NH₂, —NHR^{C}, —NR^{C}R^{D}, —OCONH₂, —OCONHR^{C}, —OCONR^{C}R^{D}, —NHCOR^{C}, —NHCOOR^{C}, —NHR^{D}COOR^{C}, —NHSO₂OR^{C}, —NR^{D}SO₂OR^{C}, —NHCONH₂, —NR^{C}CONH₂, —NHCONHR^{D}, —NR^{C} CONHR^{D}, —NHCONHR^{C}R^{D}, and —NR^{C}CONR^{C}R^{D}, wherein R^{C} and R^{D} are independently C₁-C₆ alkyl groups.

23. The method of claim 21 wherein R₂ is phenyl, optionally substituted by methyl, ethyl, n- or isopropyl, methoxy, ethoxy, isopropoxy, chloro, or bromo,
wherein the optional substituent is selected from the group consisting of: C₁-C₆ alkyl, C₁-C₆ alkoxy, hydroxyl, hydroxy C₁-C₆ alkyl, mercapto, mercapto C₁-C₆ alkyl, C₁-C₆ alkylthio, halo, trifluoromethyl, trifluoromethoxy, nitro, nitrile (—CN), oxo, phenyl, —COOH, COOR^{C}, —COR^{C}, —SO₂R^{C}, —CONH₂, —SO₂NH₂, —CONHR^{C}, —SO₂NHR^{C}, —CONR^{C}R^{D}, —SO₂NR^{C}R^{D}, —NH₂, —NHR^{C}, —NR^{C}R^{D}, —OCONH₂, —OCONHR^{C}, —OCONR^{C}R^{D}, —NHCOR^{C}, —NHCOOR^{C}, —NHR^{D}COOR^{C}, —NHSO₂OR^{C}, —NR^{D}SO₂OR^{C}, —NHCONH₂, —NR^{C}CONH₂, —NHCONHR^{D}, —NR^{C} CONHR^{D}, —NHCONHR^{C}R^{D}, and —NR^{C}CONR^{C}R^{D}, wherein R^{C} and R^{D} are independently C₁-C₆ alkyl groups.

24. The method of claim 22 wherein the optional substituent is in the 4-position of the phenyl ring.

25. The method of claim 12 wherein m is 1, and p, r and s are 0, and Q is an optionally substituted carbocyclic or heterocyclic ring,
wherein the optional substituent is selected from the group consisting of: C₁-C₆ alkyl, C₁-C₆ alkoxy, hydroxyl, hydroxy C₁-C₆ alkyl, mercapto, mercapto C₁-C₆ alkyl, C₁-C₆ alkylthio, halo, trifluoromethyl, trifluoromethoxy, nitro, nitrile (—CN), oxo, phenyl, —COOH, COOR^{C}, —COR^{C}, —SO₂R^{C}, —CONH₂, —SO₂NH₂, —CONHR^{C}, —SO₂NHR^{C}, —CONR^{C}R^{D}, —SO₂NR^{C}R^{D}, —NH₂, —NHR^{C}, —NR^{C}R^{D}, —OCONH₂, —OCONHR^{C}, —OCONR^{C}R^{D}, —NHCOR^{C}, —NHCOOR^{C}, —NHR^{D}COOR^{C}, —NHSO₂OR^{C}, —NR^{D}SO₂OR^{C}, —NHCONH₂, —NR^{C}CONH₂, —NHCONHR^{D}, —NR^{C} CONHR^{D}, —NHCONHR^{C}R^{D}, and —NR^{C}CONR^{C}R^{D}, wherein R^{C} and R^{D} are independently C₁-C₆ alkyl groups.

26. The method of claim 12 wherein Ar¹ is a phenyl or pyridyl ring.

27. The method of claim 12 wherein R₃ is amino(NH₂).

28. The method of claim 12 wherein R₄ is a carboxamide group of formula —CONR^{B}(Alk)ₙR^{A} wherein
Alk is a divalent alkylene, alkenylene or alkynylene radical, and the Alk radical may be optionally substituted,
n is 0 or 1,
R^{B} is hydrogen or a C₁-C₆ alkyl or C₂-C₆ alkenyl group,
R^{A} is hydroxy or optionally substituted carbocyclic or heterocyclyl, any of which heterocyclic rings may be substituted; or
R^{A} and R^{B} taken together with the nitrogen to which they are attached form an N-heterocyclic ring which may optionally contain one or more additional hetero atoms selected from O, S and N, and which may optionally be substituted on one or more ring C or N atoms;
wherein the optional substituent is selected from the group consisting of: C₁-C₆ alkyl, C₁-C₆ alkoxy, hydroxyl, hydroxy C₁-C₆ alkyl, mercapto, mercapto C₁-C₆ alkyl, C₁-C₆ alkylthio, halo, trifluoromethyl, trifluoromethoxy, nitro, nitrile (—CN), oxo, phenyl, —COOH, COOR^{C}, —COR^{C}, —SO₂R^{C}, —CONH₂, —SO₂NH₂, —CONHR^{C}, —SO₂NHR^{C}, —CONR^{C}R^{D}, —SO₂NR^{C}R^{D}, —NH₂, —NHR^{C}, —NR^{C}R^{D}, —OCONH₂, —OCONHR^{C}, —OCONR^{C}R^{D}, —NHCOR^{C}, —NHCOOR^{C}, —NHR^{D}COOR^{C}, —NHSO₂OR^{C}, —NR^{D}SO₂OR^{C}, —NHCONH₂, —NR^{C}CONH₂, —NHCONHR^{D}, —NR^{C}CONHR^{D}, —NHCONHR^{C}R^{D}, and —NR^{C}CONR^{C}R^{D}, wherein R^{C} and R^{D} are independently C₁-C₆ alkyl groups.

29. The composition of claim 15 wherein m is 1, each of p, r and s is 0, and Q is hydrogen.

30. The composition of claim 29 wherein R₂ is optionally substituted phenyl, 2- or 3-thienyl, 2- or 3-furanyl, or 2-, 3- or 4-pyridinyl,
wherein the optional substituent is selected from the group consisting of: C₁-C₆ alkyl, C₁-C₆ alkoxy, hydroxyl, hydroxy C₁-C₆ alkyl, mercapto, mercapto C₁-C₆ alkyl, C₁-C₆ alkylthio, halo, trifluoromethyl, trifluoromethoxy, nitro, nitrile (—CN), oxo, phenyl, —COOH, COOR^{C}, —COR^{C}, —SO₂R^{C}, —CONH₂, —SO₂NH₂, —CONHR^{C}, —SO₂NHR^{C}, —CONR^{C}R^{D}, —SO₂NR^{C}R^{D}, —NH₂, —NHR^{C}, —NR^{C}R^{D}, —OCONH₂, —OCONHR^{C}, —OCONR^{C}R^{D}, —NHCOR^{C}, —NHCOOR^{C}, —NHR^{D}COOR^{C}, —NHSO₂OR^{C}, —NR^{D}SO₂OR^{C}, —NHCONH₂, —NR^{C}CONH₂, —NHCONHR^{D}, —NR^{C} CONHR^{D}, —NHCONHR^{C}R^{D}, and —NR^{C}CONR^{C}R^{D}, wherein R^{C} and R^{D} are independently C₁-C₆ alkyl groups.

31. The composition of claim 29 wherein R₂ is phenyl, optionally substituted by methyl, ethyl, n- or isopropyl, methoxy, ethoxy, isopropoxy, chloro, or bromo,
wherein the optional substituent is selected from the group consisting of: C₁-C₆ alkyl, C₁-C₆ alkoxy, hydroxyl, hydroxy C₁-C₆ alkyl, mercapto, mercapto C₁-C₆ alkyl, C₁-C₆ alkylthio, halo, trifluoromethyl, trifluoromethoxy, nitro, nitrile (—CN), oxo, phenyl, —COOH, COOR^{C}, —COR^{C}, —SO₂R^{C}, —CONH₂, —SO₂NH₂, —CONHR^{C}, —SO₂NHR^{C}, —CONR^{C}R^{D}, —SO₂NR^{C}R^{D}, —NH₂, —NHR^{C}, —NR^{C}R^{D}, —OCONH₂, —OCONHR^{C}, —OCONR^{C}R^{D}, —NHCOR^{C}, —NHCOOR^{C}, —NHR^{D}COOR^{C}, —NHSO₂OR^{C}, —NR^{D}SO₂OR^{C}, —NHCONH₂, —NR^{C}CONH₂, —NHCONHR^{D}, —NR^{C} CONHR^{D}, —NHCONHR^{C}R^{D}, and —NR^{C}CONR^{C}R^{D}, wherein R^{C} and R^{D} are independently C₁-C₆ alkyl groups.

32. The composition of claim 30 wherein the optional substituent is in the 4-position of the phenyl ring.

33. The composition of claim 15 wherein m is 1, and p, r and s are 0, and Q is an optionally substituted carbocyclic or heterocyclic ring,
wherein the optional substituent is selected from the group consisting of: C₁-C₆ alkyl, C₁-C₆ alkoxy, hydroxyl, hydroxy C₁-C₆ alkyl, mercapto, mercapto C₁-C₆ alkyl, C₁-C₆ alkylthio, halo, trifluoromethyl, trifluoromethoxy, nitro, nitrile (—CN), oxo, phenyl, —COOH, COOR^{C}, —COR^{C}, —SO₂R^{C}, —CONH₂, —SO₂NH₂, —CONHR^{C}, —SO₂NHR^{C}, —CONR^{C}R^{D}, —SO₂NR^{C}R^{D}, —NH₂, —NHR^{C}, —NR^{C}R^{D}, —OCONH₂, —OCONHR^{C}, —OCONR^{C}R^{D}, —NHCOR^{C}, —NHCOOR^{C}, —NHR^{D}COOR^{C}, —NHSO₂OR^{C}, —NR^{D}SO₂OR^{C}, —NHCONH₂, —NR^{C}CONH₂, —NHCONHR^{D}, —NR^{C} CONHR^{D}, —NHCONHR^{C}R^{D}, and —NR^{C}CONR^{C}R^{D}, wherein R^{C} and R^{D} are independently C₁-C₆ alkyl groups.

34. The composition of claim 15 wherein Ar¹ is a phenyl or pyridyl ring.

35. The composition of claim 15 wherein R₃ is amino(NH₂).

36. The composition of claim 15 wherein R₄ is a carboxamide group of formula —CONR^{B}(Alk)ₙR^{A} wherein
Alk is a divalent alkylene, alkenylene or alkynylene radical, and the Alk radical may be optionally substituted,
n is 0 or 1,
R^{B} is hydrogen or a C₁-C₆ alkyl or C₂-C₆ alkenyl group,
R^{A} is hydroxy or optionally substituted carbocyclic or heterocyclyl, any of which heterocyclic rings may be substituted; or
R^{A} and R^{B} taken together with the nitrogen to which they are attached form an N-heterocyclic ring which may optionally contain one or more additional hetero atoms selected from O, S and N, and which may optionally be substituted on one or more ring C or N atoms;
wherein the optional substituent is selected from the group consisting of: C₁-C₆ alkyl, C₁-C₆ alkoxy, hydroxyl, hydroxy C₁-C₆ alkyl, mercapto, mercapto C₁-C₆ alkyl, C₁-C₆ alkylthio, halo, trifluoromethyl, trifluoromethoxy, nitro, nitrile (—CN), oxo, phenyl, —COOH, COOR^{C}, —COR^{C}, —SO₂R^{C}, —CONH₂, —SO₂NH₂, —CONHR^{C}, —SO₂NHR^{C}, —CONR^{C}R^{D}, —SO₂NR^{C}R^{D}, —NH₂, —NHR^{C}, —NR^{C}R^{D}, —OCONH₂, —OCONHR^{C}, —OCONR^{C}R^{D}, —NHCOR^{C}, —NHCOOR^{C}, —NHR^{D}COOR^{C}, —NHSO₂OR^{C}, —NR^{D}SO₂OR^{C}, —NHCONH₂, —NR^{C}CONH₂, —NHCONHR^{D}, —NR^{C}CONHR^{D}, —NHCONHR^{C}R^{D}, and —NR^{C}CONR^{C}R^{D}, wherein R^{C} and R^{D} are independently C₁-C₆ alkyl groups.
